(19) United States
(12) Patent Application Publication
(10) Pub. No.: US 2010/0016807 A1
(43) Pub. Date: Jan. 21, 2010

(54) Syringe with Sequentially Moving Plunger Flanges

(76) Inventor: Jacques Thilly, Rixensart (BE)

Correspondence Address: KLARQUIST SPARKMAN, LLP, 121 SW SALMON STREET, SUITE 1600, PORTLAND, OR, 97204, US

(21) Appl. No.: 12/226,235

(22) PCT Filed: Apr. 11, 2007
(86) PCT No.: PCT/EP2007/053531

§ 371 Date: Sep. 18, 2009

(30) Foreign Application Priority Data
Apr. 12, 2006 (GB) 0607401.7

(51) Int. Cl. A61M 5/315

(52) U.S. Cl. 604/218

(57) ABSTRACT

A syringe in which multiple sealing flanges are provided for sequential movement during use, to reduce sticking of the flange during use and the consequent jerking of the plunger stopper. In an embodiment longitudinally sequential flanges are linked by a flexible tube connection.

This invention relates to syringes, in particular to syringes for hypodermic injection of medicinal liquids into a patient's body. In particular this invention relates to pre-filled syringes which are provided for use pre-filled with a medicinal liquid such as a drug or vaccine solution. Such syringes normally comprise a cylindrical body made of glass or plastics material within which is located a slideably moveable plunger stopper which is moveable in the manner of a piston in an injecting direction to expel liquid content in the body through a nozzle at an injecting end to which an injection needle is connected or is connectable. The plunger stopper is normally operated by a user applying operating force to the plunger stopper via a plunger rod.

Generally the plunger stopper comprises a resilient, e.g. elastomeric, plug which is surrounded at its perimeter by plural body contacting flanges which establish a liquid-tight seal between the plunger stopper and the body. There is a problem, particularly with pre-filled syringes which are often stored for some time before use, in that the flanges can tend to stick to the inner wall of the body. Normally prior to use of a syringe the plunger stopper is moved in the injecting direction to expel the bubble of air that is often in the body. If the flanges stick, then it may be necessary to apply an excessive force to the plunger stopper to dislodge it from adhesion to the wall of the body. When the sticking between the flanges and the body is overcome this break-off force can tend to jerk the plunger stopper along the body, leading to expulsion and loss of medicinal liquid contents as well as the air bubble. Although the inner surface of the body may be lubricated, lubrication provides only a mere improvement as the initial break-off force is still substantially high relative to the sliding force, particularly for plastic material syringes. Provision of fewer flanges may reduce this problem but at the risk of an inadequate seal between the plunger stopper and body.

It is an object of this invention to address this problem and to provide a solution. Other objects of the invention will be apparent from the following description.

This invention provides a syringe comprising a cylindrical body within which is located a plunger stopper which is moveable in an injecting direction to expel liquid content in the body through a nozzle at an injecting end of the body to which an injection needle is or may be attached, with a plunger rod connected to the plunger stopper by which a user can apply an operating force to the plunger stopper to move the plunger stopper in the injecting direction along the body, characterized in that;

the plunger stopper is provided with a first body-contacting flange slideably contacting the inner surface of the body,

a hub is located in the body disposed relative to the plunger stopper in the direction opposite to the injecting direction, the plunger rod passing moveably through the hub, the hub being provided with a second body-contacting flange slideably contacting the inner surface of the body,

wherein the second body-contacting flange is moveable relative to the plunger stopper such that movement of the second body-contacting flange occurs only after the plunger stopper has begun movement toward the nozzle under the action of operating force applied to the plunger stopper by the plunger rod.

By means of the syringe of the invention, although plural first and second body-contacting flanges are present to thereby provide an adequate seal between the plunger stopper and the body, any sticking between the flanges and the inside surface of the body is overcome sequentially, that is, firstly any sticking between the first body-contacting flange and the body is overcome, so that the first body-contacting flange moves whilst the second body-contacting flange remains static, then only when the plunger stopper and the first body-contacting flange have moved is the second body-contacting flange moved.

There may be one or more first body-contacting flange. Preferably there are two first body-contacting flanges.

The syringe of the invention can be realized in various embodiments.

In a first embodiment the hub is distanced from the plunger stopper by a space along the injecting direction, and the plunger rod is provided with a surface, e.g. a shoulder part which can bear upon the hub and/or second body-contacting flange as the plunger rod moves along the body in the injecting direction so that the surface, e.g. shoulder bears upon the hub and/or second body-contacting flange to move the second body-contacting flange slideably along the body in the injecting direction,

the hub being so distanced from the plunger stopper in the direction opposite to the injecting direction that on applying operating force to the plunger stopper, the plunger stopper is first caused to move in the injecting direction, then after the plunger stopper has so moved the surface, e.g. shoulder bears upon the hub and/or second body-contacting flange to move the second body-contacting flange slideably along the body in the injecting direction.

In a preferred form of this embodiment there is a flexible tubular connection between the first and second body-contacting flanges. Such a connection may be made of an elastomeric material and may be made integrally of such a material with the first and second body engaging flanges. Suitably the flexible connection has a collapsed state in which the connection is collapsed along the injecting direction before the first body-contacting flange moves, and an extended state in which the connection is extended in the injecting direction after the first body contacting flange has moved.

In this first embodiment firstly the plunger rod moves the plunger stopper with its first body-contacting flange, whilst the second body-contacting flange remains static until the surface, e.g. shoulder contacts the hub and/or second body-contacting flange, then only when the surface, e.g. shoulder has contacted the hub and/or second body-contacting flange is the second body-contacting flange moved. When there is the above-mentioned flexible tubular connection this may communicate a pulling force from the first to the second body-contacting flange as the plunger stopper moves in the injecting direction along the body.

In a second embodiment the hub is distanced from the plunger stopper by a space, and there is a flexible tubular connection between the first and second body-contacting flanges, the flexible connection having a collapsed state in which the connection is collapsed along the injecting direction, and an extended state in which the connection is extended in the injecting direction, and in its extended state the flexible connection communicates a pulling force in the injecting direction from the first body contacting flange to the second body-contacting flange. In this embodiment the shoulder may not be necessary.

In this second embodiment, with the flexible connection in its collapsed state the plunger stopper is first caused to move in the injecting direction, then after the plunger stopper has so moved the flexible connection is thereby pulled into its extended state, and in this extended state the flexible connection communicates a pulling force in the injecting direction from the first body contacting flange to the second body-contacting flange to thereby move the second body-contacting flange slideably along the body in the injecting direction.

The body may be essentially conventional for example made of glass or plastics material.

The plunger stopper may comprise a generally conventional generally cylindrical elastomer plug and the first body-contacting flange may be integrally made with the plunger stopper. There may be plural, e.g. two, first body-contacting flanges, circumferentially surrounding the plunger stopper.

The hub suitably has an axial channel through which the plunger rod is moveably threaded. For example the hub may comprise a sleeve, e.g. toroidal sleeve, e.g. a cylinder with an axial channel parallel to the injecting direction through which the plunger rod is threaded. In an alternative construction the hub may have an axial channel which communicates with the periphery of the hub, for example so that the plunger rod may be threaded into the channel from the periphery. The hub should be sufficiently rigid that the body-contacting flange can be compressed between the body and the hub to create a liquid-tight seal. The hub is typically made of a plastics material. A suitable distance from the plunger stopper in the direction opposite to the injecting direction may be determined practically, 1-3 mm being suitable. The second body-contacting flange may circumferentially surround the hub. The dimensions of the hub and second body-contacting flange are suitably such that the second body-contacting flange slideably contacts and is compressed against the inner surface of the body to establish a liquid tight compression seal between the second body-contacting flange and the inner surface of the body.

The second body contacting flange is preferably made of an elastic material and may suitably elastically grip the hub, e.g. fitting into a circumferential groove therein, and may be integrally made with the first body-contacting flange.

The plunger rod may pass through the axial channel of such a toroidal hub with a clearance such that the plunger rod freely slideably moves in the injecting direction relative to the hub as the plunger rod drives the plunger stopper.

The plunger rod may for example be made of a plastics material. A shoulder part of the plunger rod may comprise a part of the plunger rod which is widened transverse to the injecting direction relative to an adjacent part of the plunger rod closer in the injecting direction to the nozzle. The plunger rod may be connected to the plunger by any convenient means, for example co-operating screw threads on the plunger rod and the plunger, or for example a known snap fit connection.

The flexible tubular connection between the first and second body-contacting flanges is suitably made of an elastomeric material, and may be integrally made with the first and second body-contacting flange. The flexible connection may for example be an expandible bellows-like tubular connection. The tubular connection may be such that when pulled taut it can communicate a pulling force to the hub from the first body-contacting flange.

There may be plural second body-contacting flanges. For example there may be plural hubs arranged sequentially along the body each provided with a respective second body-contacting flange. For example in an analogue of the first embodiment such plural hubs may be arranged so a first one of such plural hubs closest to the opposite end of the body from the nozzle is first moved in the injecting direction by the shoulder part of the plunger rod, then this first hub contacts a second of such plural hubs to push against that second hub to cause that second hub to move. For example a hub may be provided with plural second body-contacting flanges.

The syringe of this invention may be filled in a conventional manner. For example the body may be filled from the open end of the body opposite to the injecting end of the body. Alternatively the syringe may be filled via an opening at the injecting end of the body prior to connection of a needle thereto.

The invention will now be described by way of example with reference to the accompanying drawings.

FIG. 1 shows a longitudinal section of a syringe of the first embodiment with its flexible connector in the collapsed state.

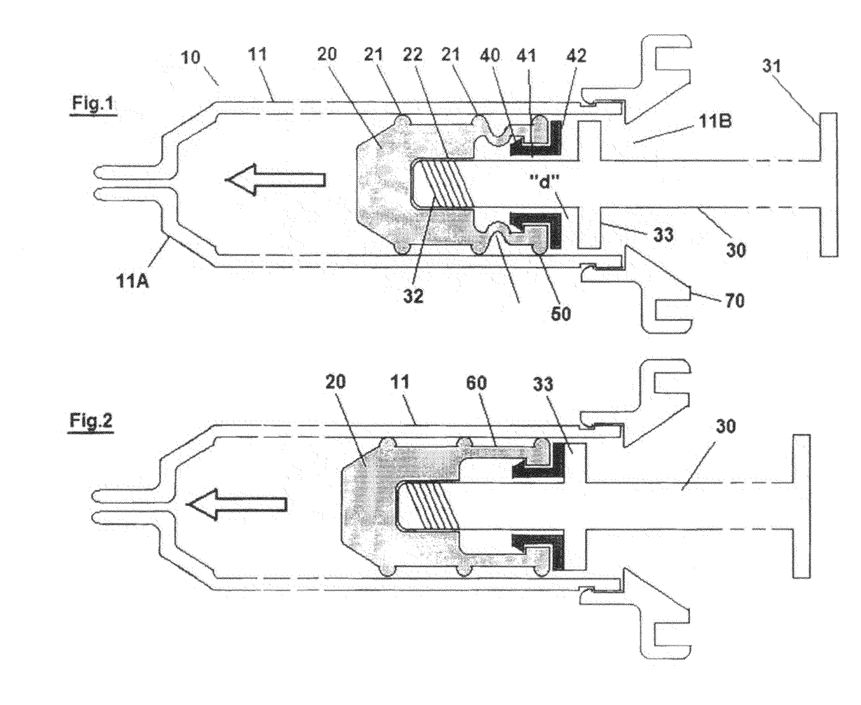

FIG. 2 shows a longitudinal section of the syringe of FIG. 1 with its flexible connector in the extended state.

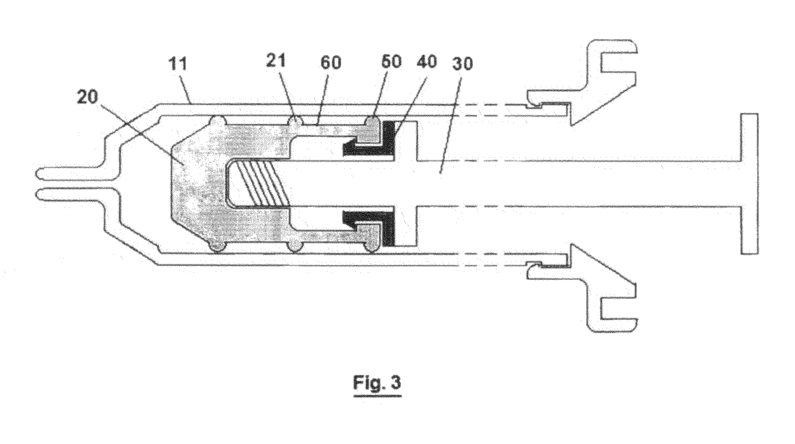

FIG. 3 shows a longitudinal section of the syringe of FIG. 2 with its plunger stopper moved further along the body.

FIGS. 4 to 7 show alternative constructions of syringes of this invention.

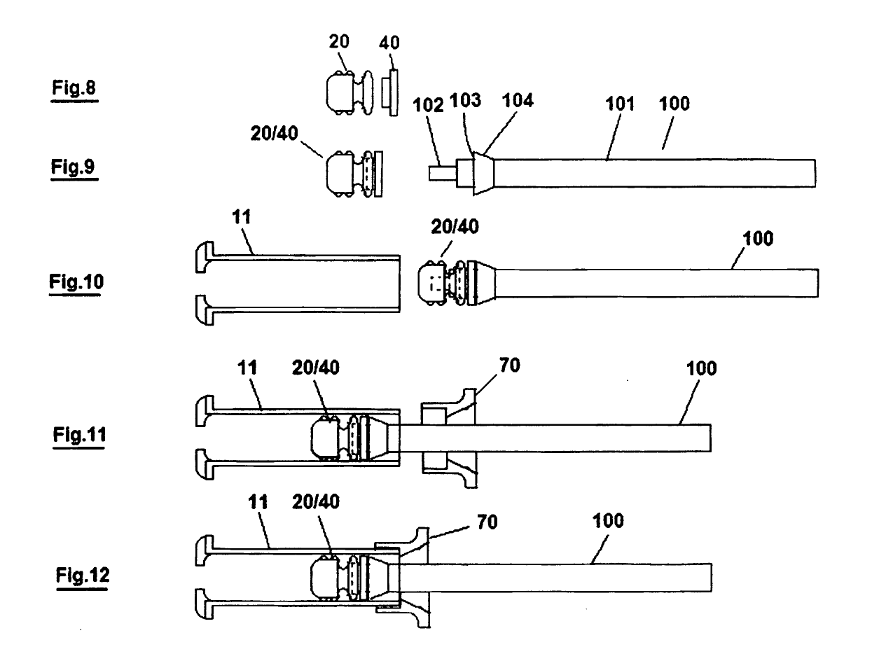

FIGS. 8 to 12 show how a syringe of this invention might be assembled.

FIGS. 13 to 17 show how the syringe of FIGS. 8 to 12 might be filled.

Referring to FIGS. 1, 2 and 3 a syringe is shown overall 10. This comprises a cylindrical body 11 having a nozzle 12 to which an injection needle (not shown) may be attached at an injecting end of the body 11A and being open at the opposite end 11B. The body 11 is made of a medically acceptable plastics material of the typical type of which syringes are made. The syringe 10 is of the pre-filled type containing a medicinal liquid (not shown) for injection to a patient through the needle. Within the body 11 is located a plunger stopper 20 which is moveable in the injecting direction 11B toward 11A (shown by the arrow) to expel liquid content through the nozzle 12. A plunger rod 30 is connected to the plunger stopper 20 by which a user can apply an operating force to the plunger stopper 20, e.g. by thumb pressure applied to button 31 to move the plunger stopper 20 in the injecting direction along the body 11.

Plunger stopper 20 comprises a generally conventional generally cylindrical elastomer plug and is provided with two first body-contacting flanges 21, though only one first body-contacting flange 21 need be used. Plunger stopper 20 and flange 21 are integrally made of the elastomer material, the flanges 21 being in the form of a ridge circumferentially surrounding plunger stopper 20 which slideably contacts the inner surface of the body 11. The plunger rod 30 connects with plunger stopper 20 by means of conventional screw threads 32 on the end of plunger rod 30 co-operating with corresponding threads in a socket 22 in plunger stopper 20, although other means of connection may be used.

A hub 40 is moveably located in the body 11 disposed relative to the plunger stopper 20 in the direction opposite to the injecting direction 11B-11A, i.e. closer to the open end 11B than the plunger stopper 20. Hub 40 comprises a toroidal member, i.e. a cylinder with an axial channel 41 parallel to the injecting direction 11B-11A, with an abutment flange 42, and is made of a plastics material. The hub 40 is at a distance “d¹” of ca. 1-3 mm from the plunger stopper in the injecting direction. Plunger rod 30 is threaded through the central channel 41 of toroidal hub 40 with a clearance such that the plunger rod 30 may freely slideably move in the injecting direction relative to the hub 40 as the plunger rod 30 drives the plunger stopper 20 in the injecting direction.

A second body-contacting flange 50 is mounted on hub 40. The flange 50 is made of an elastomeric material and fits into circumferential groove 43 in the outer surface of hub 40, circumferentially surrounding the hub 40 and elastically gripping the hub 40. The dimensions of the hub 40 and second body-contacting flange 50 are such that the second body-contacting flange 50 slideably contacts and is compressed against the inner surface of the body 11 to establish a liquid tight compression seal between the second body-contacting flange 50 and the inner surface of the body 11.

There is a flexible tubular connection 60 between the first and second body-contacting flanges 21,50. Connection 60 is made integrally of the same elastomeric material as the first and second body engaging flanges 21,50. The flanges 21,50 and connection 60 between them provide a fluid- and contamination-proof seal between the environment and the interior part 13 of body 11 where medicinal content (not shown) is contained.

As seen in FIG. 1 the flexible connection 60 is in a collapsed state in which the connection 60 is collapsed in a bellows-like manner along the injecting direction. The pre-filled syringe 10 is initially provided in the collapsed state shown in FIG. 1. The plunger rod 30 is provided with a shoulder part 33. As the plunger rod 30 is moved in the injecting direction it moves the plunger stopper 20 in the injecting direction to expel content through the nozzle 12. At this stage the plunger rod 30 moves freely through the hub 40 and the hub 40 with the second body-contacting flange 50 mounted thereon is not moved relative to the body 11. Consequently only sticking and friction between the first body-contacting flanges 21 and the body 11 needs to be overcome, and the plunger stopper 20 is easily moved without a jerk.

As seen in FIG. 2, as the plunger rod 30 continues to move in the injecting direction, pushing plunger stopper 20 in the injecting direction, the shoulder 33 bears upon the hub 40 to move the hub 40 and the second body-contacting flange 50 slideably along the body 11 in the injecting direction, and at this stage sticking and friction between the second body-contacting flange 50 and the body 11 is overcome. In this way the sticking and friction between the first and second body-contacting flanges 21, 50 and the body 11 is overcome in successive stages rather than all at the same instant.

But at the same time the plural first and second body-contacting flanges provide effective seals between the medicinal liquid content and the outside environment.

As seen in FIG. 3 on continued movement of the plunger rod 30 in the injecting direction the plunger stopper 20 is pushed further in the injecting direction toward nozzle 12 to eject liquid content from body 11 until the plunger stopper 20 abuts against the end 11A of the body, and the shoulder 33 continues to push the hub 40 in the injecting direction behind the plunger stopper 20.

FIG. 4 shows a part view of an alternative construction of the syringe of the invention. Parts corresponding to FIGS. 1, 2 and 3 are numbered correspondingly. In this construction there is no shoulder 33 on the plunger rod 30. Instead, as the plunger rod 30 moves in the injecting direction, the plunger stopper 20 with its first body-contacting flange 21 moves in the injecting direction whilst the hub 40 stays where it is whilst the flexible connection 60, initially in its collapsed state as seen in FIG. 4, analogous to FIG. 1, is pulled into its extended state. In this extended state the flexible connection 60 communicates a pulling force to the second body-contacting flange 50 to pull it and the hub 40 upon which it is mounted in the injecting direction.

FIG. 5 shows a part view of another alternative construction of the syringe of the invention. Parts corresponding to FIGS. 1, 2 and 3 are numbered correspondingly. In this construction there are plural second body-contacting flanges 50, 50′, respectively mounted on plural hubs 40,40′, with a flexible connection 60 between the first 21 and second 50 body-contacting flanges, and a flexible connection 60′ between the two second 50 body-contacting flanges, with a shoulder 33 on the plunger rod 30. This construction works analogously to the construction of FIGS. 1, 2 and 3 in that the movement of the plunger rod 30 in the injecting direction causes the shoulder 33 to first contact the hub 40′ and cause that to move, then hub 40′ contacts hub 40 to move.

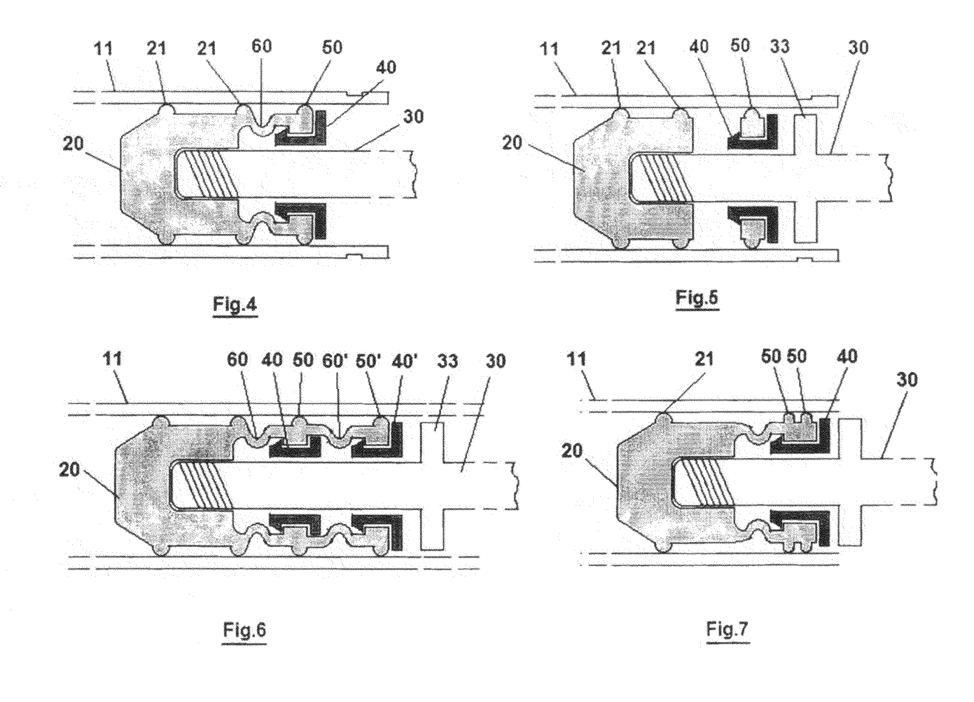

FIG. 6 shows a part view of another alternative construction of the syringe of the invention, in which there are plural second body-contacting flanges 50 on hub 40, and a single first body-contacting flange 21 on the plunger stopper 20. This syringe works analogously to the syringe of FIGS. 1, 2 and 3.

On the syringe body 11 of FIGS. 1, 2 and 3 a back stop/finger grip 70 is provided to prevent the plunger stopper 20 being pulled out of the open end 11B of body 11 in the direction opposite to the injecting direction. Back stop 70 engages with the body 11 in a conventional snap-fit engagement. The relative dimensions of the shoulder 33 and the opening 71 in backstop 70 are such that the backstop 70 prevents removal of the shoulder from the body 11.

FIGS. 7 to 11 show how a syringe of FIGS. 1 to 3 might be assembled.

Referring to FIG. 7 a plunger stopper 20 and a hub 40 are shown adjacent but unassembled.

Referring to FIG. 8 a plunger stopper 20 and a hub 40 are shown assembled, with a tool 100 adjacent. The tool 100 has a shaft 101 with an end 102 of analogous shape to the end of the plunger rod 30 of the syringe of FIGS. 1 to 3, to fit into the socket 22 in the plunger stopper 20, but without screw threads, with a shoulder 103 to abut against the hub 40.

Referring to FIG. 9 the end 102 has been threaded through hub 40 and inserted into the socket 22 in plunger stopper 20.

Referring to FIG. 10, the assembly of tool 100, plunger stopper 20 and hub 40 has been inserted into the open end 11B of syringe body 11. A back stop 70 has been threaded over the end of the plunger rod 101 of tool 100.

Referring to FIG. 11 the back stop 70 has been snap-fit attached to the body 11. In FIGS. 7 to 11 the flexible connection 60 is in its collapsed, unstretched configuration.

The body 11 seen in FIGS. 8-16 is provided with a front stopper 300, which closes the open end of the body 11 at its injecting end 11A, and has an opening 301 via which the body 11 may be filled with its liquid content (not shown) and a needle (not shown) attached.

FIGS. 12 to 16 show a possible filling operation for the syringe of FIGS. 1 and 2.

Referring to FIG. 12 the tool 100 has been withdrawn from the body 11. The tool 100 is provided with a conical ramp surface 104 which enables the tool 100 to pass back stop 70. This leaves the assembly of plunger stopper 20 and hub 40 in place in the body 11 as seen in FIG. 12. The body 11 has been oriented vertically, with the opening 301 uppermost for filing. In this orientation the body 11 may be sterilized, e.g. by means of an electron beam or other method, and may be vented.

Referring to FIG. 13 a second tool 200, analogous to the first tool 100 may if necessary be inserted through back stop 70 to engage the assembly of plunger stopper 20 and hub 40 to adjust the assembly 20,40 if necessary to a position along body 11 appropriate for the fill volume. Tool 200 also has a conical ramp surface 204 to enable it to be withdrawn through backstop 70.

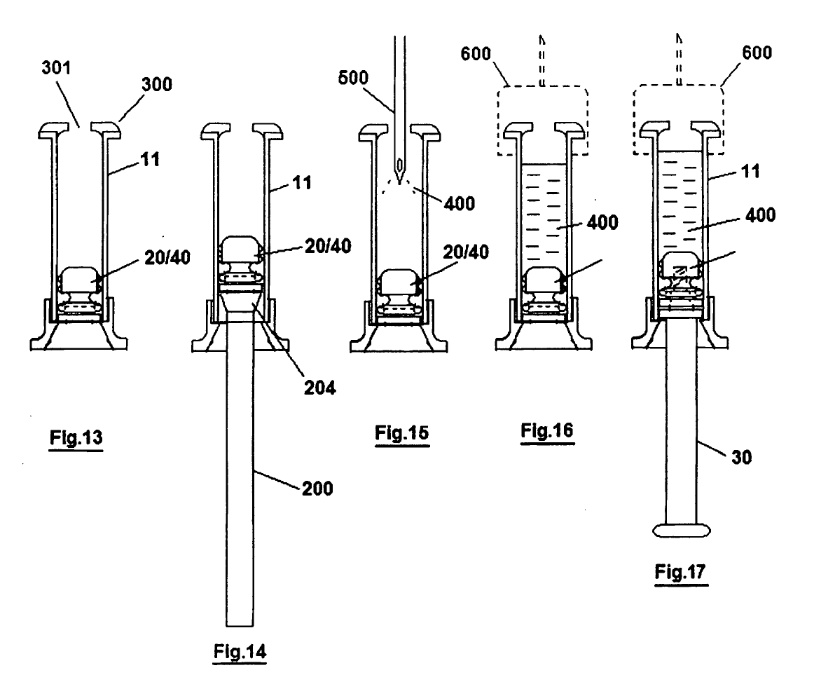

Referring to FIG. 14 the body 11 may now be filled with liquid content 400 using filling needle 500 inserted through the opening 301. After a suitable volume of liquid content has been introduced into the body 11 the tool 200 (not shown in FIG. 14) may be used to adjust the assembly 20,40 if necessary to a position along body 11 appropriate for the fill volume, i.e. to expel air through opening 301 to reduce the volume of any residual bubble remaining in body 11.

Referring to FIG. 15 the body 11 has now been filled with an appropriate volume of liquid 400 and the assembly 20,40 is at an appropriate position along the body. An injection needle mounting 600 (shown schematically) is now assembled to the end 11A of the body 11.

Referring to FIG. 16 a plunger rod 30 has now been threaded through hub 40 and connected to piston 20, so that the syringe is ready for use.

It will be appreciated that an analogous procedure may be used to fill the body 11 via the open end 11B of the body before the plunger and hub assembly 20/40 is introduced into body 11. It will be apparent that to do so the body 11 needs to be in a configuration which is inverted relative to the configuration shown I FIGS. 12 to 17.

The assembly and filling operations as described with reference to FIGS. 8 to 16 are preferably performed in a sterile environment, e.g. under a conventional downward stream of sterile filtered air.

1. A syringe comprising:
a cylindrical body within which is located a plunger stopper which is moveable in an injecting direction to expel liquid content in the body through a nozzle at an injecting end of the body to which an injection needle is or may be attached, with a plunger rod connected to the plunger stopper by which a user can apply an operating force to the plunger stopper to move the plunger stopper in the injecting direction along the body, wherein the plunger stopper is provided with a first body-contacting flange slideably contacting the inner surface of the body, a hub is located in the body disposed relative to the plunger stopper in the direction opposite to the injecting direction, the plunger rod passing moveably through the hub, the hub being provided with a second body-contacting flange slideably contacting the inner surface of the body, the second body-contacting flange being moveable relative to the plunger stopper, wherein the plunger rod comprises a shoulder which is provided to bear upon the hub and/or second body-contacting flange to move the second body-contacting flange as the plunger rod moves in the injecting direction, such that the shoulder bears upon the hub and/or second body-contacting flange only after the plunger stopper has begun movement toward the nozzle under action of operating force applied to the plunger stopper by the plunger rod and, as the plunger rod continues to move in the injecting direction, movement of the second body-contacting flange occurs, and wherein a flexible tubular connection is provided between the first and second body-contacting flanges so that the flexible tubular connection and the first and second body-contacting flanges provide a fluid- and contamination-proof seal between the content in the body and the environment.

2. (canceled)

3. (canceled)

4. The syringe of claim 1 wherein the flexible connection is made of an elastomeric material integrally with the first and second body engaging flanges.

5. The syringe of claim 3 wherein the flexible connection has a collapsed state in which the connection is collapsed along the injecting direction, and an extended state in which the connection is extended in the injecting direction.

6. The syringe of claim 1 wherein, in operation, firstly the plunger rod moves the plunger stopper with its first body-contacting flange, whilst the second body-contacting flange remains static until the shoulder contacts the hub and/or second body-contacting flange, then only when the shoulder has contacted the hub and/or second body-contacting flange is the second body-contacting flange moved.

7. The syringe of claim 6 wherein the flexible tubular connection between the first and second body-contacting flanges communicates a pulling force from the first to the second body-contacting flanges as the plunger stopper moves in the injecting direction along the body.

8. (canceled)

9. (canceled)

10. The syringe of claim 1 wherein that tubular flexible connection is an expandible bellows-like tubular connection.

11. The syringe of claim 1 wherein the tubular connection is stretchable so that when stretched taut it can communicate a pulling force to the hub from the first body-contacting flange.

12. The syringe of claim 1 wherein plural first body-contacting flanges circumferentially surround the plunger stopper.

13. The syringe of claim 1 wherein the hub comprises a toroidal sleeve in the form of a cylinder with an axial channel parallel to the injecting direction.

14. The syringe of claim 1 wherein the hub is sufficiently rigid that the second body-contacting flange is compressed between the body and the hub to create a liquid-tight seal.

15. The syringe of claim 1 wherein the second body contacting flange is made of an elastic material and elastically grips the hub.

16. The syringe of claim 13, wherein the plunger rod passes through the axial channel of the toroidal hub with a clearance such that the plunger rod freely slideably moves in the injecting direction relative to the hub as the plunger rod drives the plunger stopper.
